(19) United States
(12) Patent Application Publication
(10) Pub. No.: US 2022/0235360 A1
(43) Pub. Date: Jul. 28, 2022

(54) METHODS FOR MODULATING IMMUNOGLOBULIN EXPRESSION

(71) Applicants: INSERM (INSTITUT NATIONAL DE LA SANTÉ ET DE LA RECHERCHE MÉDECALE), Paris (FR); CENTRE NATIONAL DE LA RECERCHE SCIENTIFIQUE (CNRS), Paris (FR); CENTRE HOSPITAL RÉGIONAL UNIVERSITAIRE DE LIMOGES, Limoges (FR); UNIVERSITÉ DE LIMOGES, Limoges (FR)

(72) Inventors: Laurent DELPY, Limoges (FR); Michel COGNÉ, Limoges (FR); Brice LAFFLEUR, New-York, NY (US); Anne MARCHALOT, Limoges (FR)

(21) Appl. No.: 17/615,621

(22) PCT Filed: Jun. 3, 2020
(86) PCT No.: PCT/EP2020/065327

§ 371 Date: Dec. 1, 2021

(30) Foreign Application Priority Data
Jun. 4, 2019 (EP) 19305716.3

(51) Int. Cl. C12N 15/113

(52) U.S. Cl. CPC C12N 15/1138; C12N 2310/3233; C12N 2310/11

(57) ABSTRACT

Immunoglobulins (Ig) are expressed either on the surface of B cells or as secreted antibodies by plasma cells that represents the final stage of B cell differentiation. The present invention involves the use of antisense oligonucleotides (ASOs) for either reducing the production of the secreted form or either reducing the production of the membrane form. In particular, the inventors show that antisense oligonucleotides masking the secretory polyadenylation signal induce a decrease in the production of the secreted immunoglobulin. Inversely, antisense oligonucleotides masking the membrane polyadenylation signal induce a decrease in the production of the membrane-anchored immunoglobulin. The proof of concept has been obtained using an ASO hybridizing to the polyadenylation signal (PAS) sequence of the transcript encoding the secreted form of IgE. Indeed, the targeting of this PAS sequence induces a drastic decrease in IgE production. Thus the choice of the right antisense oligonucleotide would be suitable for the treatment of diseases associated to B-cell development (e.g. autoimmune diseases, inflammation or B-cell malignancies).

### FIELD OF THE INVENTION

The present invention is in the field of medicine, in particular immunology.

### BACKGROUND OF THE INVENTION

Immunoglobulins are molecules produced by activated B cells and plasma cells in response to exposure to antigens. There are five immunoglobulin classes (isotypes) of antibody molecules found in serum: IgG, IgM, IgA, IgE and IgD. They are distinguished by the type of heavy chain they contain. IgG molecules possess heavy chains known as γ-chains; IgM have chains; IgA have α-chains; IgE have ε-chains; and IgD have δ-chains. The variation in heavy chain polypeptides allows each immunoglobulin class to function in a different type of immune response or during a different stage of the body's defense. Upon antigen exposure, these molecules are secreted allowing the immune system to recognize and effectively respond to a myriad of pathogens. Immunoglobulin or antibody secreting cells are the mature form of B lymphocytes, which during their development undergo gene rearrangements and selection in the bone marrow ultimately leading to the generation of B cells, each expressing a single antigen-specific receptor/immunoglobulin molecule. Each individual immunoglobulin molecule has an affinity for a unique motif, or epitope, found on a given antigen. When presented with an antigen, activated B cells differentiate into either plasma cells (which secrete large amounts of antibody that is specific for the inducing antigen), or memory B cells (which are long-lived and elicit a stronger and faster response if the host is re-exposed to the same antigen). The secreted form of immunoglobulin, when bound to an antigen, serves as an effector molecule that directs other cells of the immune system to facilitate the neutralization of soluble antigen or the eradication of the antigen-expressing pathogen. The immunoglobulin gene encodes both membrane-associated and secreted proteins through alternative RNA processing reactions. This gene indeed contains competing cleavage-polyadenylation and RNA splicing reactions and the relative use of the two pathways is differentially regulated between B cells and plasma cells. More particularly, one mRNA is cleaved and polyadenylated at an upstream poly(A) signal while the other mRNA removes this poly(A) signal by RNA splicing and is cleaved and polyadenylated at a downstream poly(A) site. General cleavage-polyadenylation and RNA splicing reactions are tightly regulated during B cell maturation to affect immunoglobulin expression. Regulation of the production of secreted immunoglobulins is highly important for an effective immune response and dysregulation of immunoglobulin production is characteristic of several antibody-mediated diseases. Inversely, regulation of the production of membrane-anchored immunoglobulins would be suitable for the treatment of B-cell lymphomas by reducing the survival signaling induced by the BCR in malignant B cells.

### SUMMARY OF THE INVENTION

As defined by the claims, the present invention relates to methods for modulating immunoglobulin expression in subjects in need thereof.

### DETAILED DESCRIPTION OF THE INVENTION

Immunoglobulins (Ig) are expressed either on the surface of B cells or as secreted antibodies by plasma cells that represents the final stage of B cell differentiation. The present invention involves the use of antisense oligonucleotides (ASOs) for either reducing the production of the secreted form or either reducing the production of the membrane form. In particular, the inventors show that antisense oligonucleotides masking the secretory polyadenylation signal induce a decrease in the production of the secreted immunoglobulin. Inversely, antisense oligonucleotides masking the membrane polyadenylation signal induce a decrease in the production of the membrane-anchored immunoglobulin. The proof of concept has been obtained using an ASO hybridizing to the polyadenylation signal (PAS) sequence of the transcript encoding the secreted form of IgE. Indeed, the targeting of this PAS sequence induces a drastic decrease in IgE production. Thus the choice of the right antisense oligonucleotide would be suitable for the treatment of diseases associated to B-cell development (e.g. autoimmune diseases, inflammation or B-cell malignancies).

Accordingly, the first object of the present invention relates to a method of modulating the expression of an immunoglobulin in a subject in need thereof comprising administering to the subject an effective amount of:
an antisense oligonucleotide complementary to a sequence comprising the secretory polyadenylation signal within the pre-mRNA molecule encoding for the immunoglobulin heavy chain for reducing the production of the secreted immunoglobulin or
an antisense oligonucleotide complementary to a sequence comprising the membrane-anchored specific polyadenylation signal within the pre-mRNA molecule encoding for the immunoglobulin heavy chain for reducing the production of the membrane-anchored immunoglobulin.

As used herein, the term “immunoglobulin” or “antibody” has its general meaning in the art and refers to a glycoprotein composed of one or more units, each containing four polypeptide chains: two identical heavy chains (H) and two identical light chains (L). The amino terminal ends of the polypeptide chains show considerable variation in amino acid composition and are referred to as the variable (V) regions to distinguish them from the relatively constant (C) regions. Each L chain consists of one variable domain, VL, and one constant domain, CL. The H chains consist of a variable domain, VH, and 3-4 constant domains CH1, CH2, CH3 and optionally CH4 depending of the Ig subclass. Each heavy chain has about twice the number of amino acids and molecular weight (˜50,000) as each light chain (˜25,000), resulting in a total immunoglobulin monomer molecular weight of approximately 150,000. The term “immunoglobulin” encompasses “membrane-anchored immunoglobulins” as well as “secreted immunoglobulins”. Membrane-anchored or membrane-bound immunoglobulins are also termed surface immunoglobulins, which are generally part of the BCR. There are five immunoglobulin classes (isotypes) of antibody molecules found in serum: IgG, IgM, IgA, IgE and IgD.

In some embodiments, the method of the present is particularly suitable for modulating the expression of IgG immunoglobulins.

As used herein, the term “IgG” has its general meaning in the art and refers to an immunoglobulin that possesses heavy γ-chains. Produced as part of the secondary immune response to an antigen, this class of immunoglobulin constitutes approximately 75% of total serum Ig. IgG is the only class of Ig that can cross the placenta in humans, and it is largely responsible for protection of the newborn during the first months of life. IgG is the major immunoglobulin in blood, lymph fluid, cerebrospinal fluid and peritoneal fluid and a key player in the humoral immune response. Serum IgG in healthy humans presents approximately 15% of total protein beside albumins, enzymes, other globulins and many more. There are four IgG subclasses described in human, mouse and rat (e.g. IgG1, IgG2, IgG3, and IgG4 in humans). The subclasses differ in the number of disulfide bonds and the length and flexibility of the hinge region. Except for their variable regions, all immunoglobulins within one class share about 90% homology, but only 60% among classes.

IgG1 comprises 60 to 65% of the total main subclass IgG, and is predominantly responsible for the thymus-mediated immune response against proteins and polypeptide antigens. IgG1 binds to the Fc-receptor of phagocytic cells and can activate the complement cascade via binding to C1 complex. IgG1 immune response can already be measured in newborns and reaches its typical concentration in infancy.

IgG2, the second largest of IgG isotypes, comprises 20 to 25% of the main subclass and is the prevalent immune response against carbohydrate/polysaccharide antigens. “Adult” concentrations are usually reached by 6 or 7 years old.

IgG3 comprises around 5 to 10% of total IgG and plays a major role in the immune responses against protein or polypeptide antigens. The affinity of IgG3 can be higher than that of IgG1.

Comprising usually less than 4% of total IgG, IgG4 does not bind to polysaccharides. In the past, testing for IgG4 has been associated with food allergies, and recent studies have shown that elevated serum levels of IgG4 are found in patients suffering from sclerosing pancreatitis, cholangitis and interstitial pneumonia caused by infiltrating IgG4 positive plasma cells.

The properties of IgG are:
Molecular weight: 150,000
H-chain type (MW): gamma (53,000)
Serum concentration: 10 to 16 mg/mL
Percent of total immunoglobulin: 75%
Glycosylation (by weight): 3%
Distribution: intra- and extravascular
Function: secondary response

In some embodiments, the method of the present is particularly suitable for modulating the expression of IgA immunoglobulins.

As used herein, the term “IgA” has its general meaning in the art and refers to an immunoglobulin that possesses heavy α-chains. IgA comprises approximately 15% of all immunoglobulins in healthy serum. IgA in serum is mainly monomeric, but in secretions, such as saliva, tears, colostrums, mucus, sweat, and gastric fluid, IgA is found as a dimer connected by a joining peptide. Most IgA is present in secreted form. This is believed to be due to its properties in preventing invading pathogens by attaching and penetrating epithelial surfaces. IgA is a very weak complement-activating antibody; hence, it does not induce bacterial cell lysis via the complement system. However, secretory IgA works together with lysozymes (also present in many secreted fluids), which can hydrolyze carbohydrates in bacterial cell walls thereby enabling the immune system to clear the infection. IgA is predominantly found on epithelial cell surfaces where it acts as a neutralizing antibody. Two IgA subtypes exist in humans, IgA1 and IgA2, while mice have only one subclass. They differ in the molecular mass of the heavy chains and in their concentration in serum. IgA1 comprises approximately 85% of total IgA concentration in serum. Although IgA1 shows a broad resistance against several proteases, there are some that can affect/splice on the hinge region. IgA1 shows a good immune response to protein antigens and, to a lesser degree, polysaccharides and lipopolysaccharides. IgA2, representing only up to 15% of total IgA in serum, plays a crucial role in the mucosa of the airways, eyes and the gastrointestinal tract to fight against polysaccharide and lipopolysaccharide antigens. It also shows good resistance to proteolysis and many bacterial proteases, supporting the importance of IgA2 in fighting bacterial infections.

Properties of IgA are:
Molecular weight: 320,000 (secretory)
H-chain type (MW): alpha (55,000)
Serum concentration: 1 to 4 mg/mL
Percent of total immunoglobulin: 15%
Glycosylation (by weight): 10%
Distribution: intravascular and secretions
Function: protect mucus membranes

In some embodiments, the method of the present is particularly suitable for modulating the expression of IgM immunoglobulins.

As used herein, the term “IgM” has its general meaning in the art and refers to an immunoglobulin that possesses heavy μ-chains. Serum IgM exists as a pentamer in mammals and comprises approximately 10% of normal human serum Ig content. It predominates in primary immune responses to most antigens and is the most efficient complement-fixing immunoglobulin. IgM is also expressed on the plasma membrane of B lymphocytes as a monomer. In this form, it is a B cell antigen receptor, with the H chains each containing an additional hydrophobic domain for anchoring in the membrane. Monomers of serum IgM are bound together by disulfide bonds and a joining (J) chain. Each of the five monomers within the pentamer structure is composed of two light chains (either kappa or lambda) and two heavy chains. Unlike in IgG (and the generalized structure shown above), the heavy chain in IgM monomers is composed of one variable and four constant regions, with the additional constant domain replacing the hinge region. IgM can recognize epitopes on invading microorganisms, leading to cell agglutination. This antibody-antigen immune complex is then destroyed by complement fixation or receptor-mediated endocytosis by macrophages. IgM is the first immunoglobulin class to be synthesized by the neonate and plays a role in the pathogenesis of some autoimmune diseases. Immunoglobulin M is the third most common serum Ig and takes one of two forms:
a pentamer where all heavy chains are identical and all light chains are identical
a monomer (e.g., found on B lymphocytes as B cell receptors) IgM is the first antibody built during an immune response. It is responsible for agglutination and cytolytic reactions since in theory, its pentameric structure gives it 10 free antigen-binding sites as well as it possesses a high avidity. Due to conformational constraints among the 10 Fab portions, IgM only has a valence of 5. Additionally, IgM is not as versatile as IgG. However, it is of vital importance in complement activation and agglutination. IgM is predominantly found in the lymph fluid and blood and is a very effective neutralizing agent in the early stages of disease. Elevated levels can be a sign of recent infection or exposure to antigen.

Properties of IgM are:
Molecular weight: 900,000
H-chain type (MW): mu (65,000)
Serum concentration: 0.5 to 2 mg/mL
Percent of total immunoglobulin: 10%
Glycosylation (by weight): 12%
Distribution: mostly intravascular
Function: primary response

In some embodiments, the method of the present is particularly suitable for modulating the expression of IgE immunoglobulins.

As used herein, the term “IgE” has its general meaning in the art and refers to an immunoglobulin that possesses heavy ε-chains. The heavy chain of IgE contains an extra domain, by which it attaches with high affinity to Fc epsilon Receptor I (FcεRI) found primarily on eosinophils, mast cells and basophils. When antigens such as pollen, venoms, fungus, spores, dust mites or pet dander bind with the Fab portion of the IgE attached to the cells, the cells degranulate and release factors like heparin, histamine, proteolytic enzymes, leukotrienes and cytokines. As a consequence, vasodilatation and increased small vessel permeability causes fluid to escape from capillaries into the tissues, leading to the characteristic symptoms of an allergic reaction. Most of these typical allergic reactions like mucus secretion, sneezing, coughing or tear production are considered beneficial to expel remaining allergens from the body. Studies have shown that conditions such as asthma, rhinitis, eczema, urticaria, dermatitis and some parasitic infections (e.g., helminths and tapeworms) lead to increased IgE levels. Binding of eosinophils with Fc receptors to IgE-coated parasitic helminth worms results in death of the parasite.

Properties of IgE are:
Molecular weight: 200,000
H-chain type (MW): epsilon (73,000)
Serum concentration: 10 to 400 ng/mL
Percent of total immunoglobulin: 0.002%
Glycosylation (by weight): 12%
Distribution: basophils and mast cells in saliva and nasal secretions
Function: protect against parasites

As used herein, the expression “reducing the production of a [secreted or membrane] immunoglobulin” means a measurable decrease in the number of said immunoglobulin either in the serum or in the membrane. The reduction can be at least about 10%, e.g., at least about 20%, 30%, 40%, 50%, 60%, 70%, 80%, 90%, 95%, 96%, 97%, 98%, 99%, or more. In some embodiments, the term refers to a decrease in the number of said immunoglobulin to an amount below detectable limits. Methods for quantifying expression of immunoglobulins are well known in the art (see e.g. Normansell, David E., and L. M. Killingsworth. “Quantitation of serum immunoglobulins.” CRC Critical Reviews in Clinical Laboratory Sciences 17.2 (1982): 103-170).

As used herein, the term “polyadenylation signal” or “PAS” has its general meaning in the art and refers to a particular region of the gene encoding for the immunoglobulin heavy chain. Said gene comprises two polyadenylation signals: the secretory specific polyadenylation signal (“pAS”) and the membrane-anchored specific polyadenylation signal (“pAM”). Indeed for all genes encoding immunoglobulin heavy chains, there is an exon in the constant region that is either spliced at an internal 5′ splice site or cleaved and polyadenylated at the secretory-specific poly(A) signal (pAS). When the pre-mRNA is cleaved at the pAS, the mRNA encodes the secretory immunoglobulin. When the pre-mRNA is spliced to one or two downstream exons and the downstream poly(A) signal (pAM or membrane-anchored specific poly(A)) is used, the mRNA encodes the membrane-anchored immunoglobulin. More specifically, the pAS signal is preferably about 100-150 nucleotides downstream of the last constant region exon which encodes the 3′ end of the secretory-specific mRNA. The 3′ end of membrane-anchored specific mRNA is encoded by a large portion of the last constant region and by two downstream exons, M1 and M2. Membrane-anchored specific mRNA is produced when splicing of the last constant region exon to M1 takes place using the internal 5′ splice site within the last constant region exon, polyadenylation occurs at the pAM site at the end of M2, and preferably the intronic sequence between the M1 and M2 exons is also spliced.

As well-known from the skilled person, the highly conserved polyadenylation signal sequence 5′-ANUAAA-3′ (SEQ ID NO:1) is typically embedded in an AU-rich region (28 of 29 nucleotides surrounding the ANUAAA are A or U) and wherein N is A excepting for the pAM signal for IgE wherein N is G. The polyadenylation signal preferably contains two downstream GU-rich sequences that are located at 1 and 38 nucleotides from the cleavage site.

FIGS. 4 and 5 shows the different secretory and membrane polyadenylation signals (pAS and pAM respectively) for IgG, IgA, IgM and IgE classes.

As used herein, the term “antisense oligonucleotide” or ASO refers to a single strand of DNA, RNA, or modified nucleic acids that is complementary to a chosen sequence. Antisense RNA can be used to prevent protein translation of certain mRNA strands by binding to them. Antisense DNA can be used to target a specific, complementary (coding or non-coding) RNA. In some embodiments, the antisense oligonucleotide of the present invention is an antisense RNA. In some embodiments, the antisense oligonucleotide of the present invention is an antisense DNA.

As used herein, the term “complementary” as used herein includes “fully complementary” and “substantially complementary”, meaning there will usually be a degree of complementarity between the oligonucleotide and its corresponding target sequence of more than 80%, preferably more than 85%, still more preferably more than 90%, most preferably more than 95%. For example, for an oligonucleotide of 20 nucleotides in length with one mismatch between its sequence and its target sequence, the degree of complementarity is 95%.

In some embodiments, the antisense oligonucleotide of the present invention comprises the 5′-TTTANT-3′ sequence (SEQ ID NO:2) wherein N is T and can be exceptionally C (i.e. for targeting the pAM for IgE) or the 5′-UUUANU-3′ sequence (SEQ ID NO:3) wherein N is U and can be exceptionally C (i.e. for targeting the pAM for IgE).

In some embodiments, the antisense oligonucleotide of the present invention has a length of at least 15 nucleotides. The optimal length of the ASO's for a targeted complementary sequence is generally in the range of from about 15 to about 30 nucleotides long depending on the chemical backbone used and on the target sequence. Thus, in some embodiments, the antisense oligonucleotide has a length of at least 15, 16, 17, 18, 19, 20, 21, 22, 23, 24, 25, 26, 27, 28, 29, or 30 nucleotides. Typically, morpholino-ASOs are about 25 nucleotides long, 2′modified-ASOs are about 20 nucleotides long, and tricyclo-ASOs are about 15 nucleotides long.

For reducing the production of the secreted form, the antisense oligonucleotide of the present invention is designed to be complementary to a sequence comprising the secretory polyadenylation signal within the pre-mRNA molecule encoding for the immunoglobulin heavy chain (FIG. 1A). The antisense oligonucleotide thus will sterically hinder the secretory polyadenylation signal during the splicing reaction so as to promote the use of the alternative membrane-anchored specific polyadenylation signal encoding the membrane form of the corresponding immunoglobulin (FIG. 1A). In some embodiments, the antisense oligonucleotide is thus complementary to a sequence comprising the secretory polyadenylation signal within the sequences depicted in FIG. 4.

In some embodiments, the antisense oligonucleotide is complementary to the sequence as set forth in SEQ ID NO:4, SEQ ID NO:5, SEQ ID NO:6, SEQ ID NO:7, SEQ ID NO:8, SEQ ID NO:9, SEQ ID NO:10, or SEQ ID NO:11.

>PAS_IGHA1
SEQ ID NO: 4
CCCGCCTGTCCCCACCCCTGAATAAACTCCATGCTCCCCCAAGCAG
>PAS_IGHA2
SEQ ID NO: 5
CCCGCCTGTCCCCACCCCTGAATAAACTCCATGCTCCCCCAAGCAG
>PAS_IGHG1
SEQ ID NO: 6
TCCCAGGCACCCAGCATGGAAATAAAGCACCCAGCGCTTCCCTGGG
>PAS_IGHG2
SEQ ID NO: 7
TCCCGGGCACCCAGCATGGAAATAAAGCACCCAGCGCTGCCCTGGG
>PAS_IGHG3
SEQ ID NO: 8
TCCCGGGCACCCAGCATGGAAATAAAGCACCCAGCGCTGCCCTGGG
>PAS_IGHG4
SEQ ID NO: 9
TCCCGGGCGCCCAGCATGGAAATAAAGCACCCAGCGCTGCCCTGGG
>PAS_IGHM
SEQ ID NO: 10
TTGCATCTTATAAAATTAGAAATAAAAAGATCCATTCAAAAGATAC
>PAS_IGHE
SEQ ID NO: 11
GACCCCAGGAAGCTACCCCCAATAAACTGTGCCTGCTCAGAGCCCC

In some embodiments, for reducing the production of the secreted immunoglobulin, the antisense oligonucleotide of the present invention, comprising a nucleic acid sequence selected from the group consisting of SEQ ID NO:12, SEQ ID NO:13, SEQ ID NO:14 and SEQ ID NO:15.

In some embodiments, an antisense oligonucleotide comprising a nucleic acid sequence as set forth in SEQ ID NO:12 is used for reducing the production of secreted IgM immunoglobulins.

In some embodiments, an antisense oligonucleotide comprising a nucleic acid sequence as set forth in SEQ ID NO:13 is used for reducing the production of secreted IgG immunoglobulins.

In some embodiments, an antisense oligonucleotide comprising a nucleic acid sequence as set forth in SEQ ID NO:14 is used for reducing the production of secreted IgE immunoglobulins.

In some embodiments, an antisense oligonucleotide comprising a nucleic acid sequence as set forth in SEQ ID NO:15 is used for reducing the production of secreted IgA immunoglobulins.

>IgM-PAS:
SEQ ID NO: 12
5′-CTTTTGAATGGATCTTTTTATTTCT
>IgG-PAS:
SEQ ID NO: 13
5′-GCGCTGGGTGCTTTATTTCCATG
>IgE-PAS:
SEQ ID NO: 14
5′-GCTCTGAGCAGGCACAGTTTATTG
>IgA-PAS:
SEQ ID NO: 15
5′-GGGAGCATGGAGTTTATTCA

For reducing the production of the membrane form, the antisense oligonucleotide of the present invention is designed to be complementary to a sequence comprising the membrane-anchored specific polyadenylation signal within the pre-mRNA molecule encoding for the immunoglobulin heavy chain. The antisense oligonucleotide thus will sterically hinder the membrane-anchored specific polyadenylation signal during the splicing reaction so as to promote the use of the alternative secretory specific polyadenylation signal encoding the secreted form of the corresponding immunoglobulin. In some embodiments, the antisense oligonucleotide is thus complementary to a sequence comprising the membrane-anchored specific polyadenylation signal within the sequences depicted in FIG. 5.

In some embodiments, the antisense oligonucleotide is complementary to the sequence as set forth in SEQ ID NO:16, SEQ ID NO:17, SEQ ID NO:18, SEQ ID NO:19, SEQ ID NO:20, SEQ ID NO:21, SEQ ID NO:22, or SEQ ID NO:23.

>PAM_IGHA1
SEQ ID NO: 16
CTCACATGCCTTCCAGGTGCAATAAAGTGGCCCCAAGGAAAATGTT
>PAM_IGHA2
SEQ ID NO: 17
CTCACGTGGCTTCCAGGTGCAATAAAGTGGCCCCAAGGAAAATGTT
>PAM_IGHG1
SEQ ID NO: 18
GATGTTTCTTTTGTGATGACAATAAAATATCCTTTTTAAGTCTTGT
>PAM_IGHG2
SEQ ID NO: 19
GATGTTTCTTTTGTGATGACAATAAAATATCCTTTTTAAGTCTTGT
>PAM_IGHG3
SEQ ID NO: 20
GATGTTTCTTTTGTGATGACAATAAAATATCCTTTTTAAGTCTTGT
>PAM_IGHG4
SEQ ID NO: 21
GATGTTTCTTTTGTGATGACAATAAAATATCCTTTTTAAGTCTTGT
>PAM_IGHM
SEQ ID NO: 22
GTATACGCTTGTTGCCCTGAAATAAATATGCACATTTTATCCATGA
>PAM_IGHE
SEQ ID NO: 23
TCTTTCTCTCTGGGTTTCTTAGTAAAGATCCTTTTCACAAACCCCA

In some embodiments, for reducing the production of the membrane-anchored immunoglobulin, the antisense oligonucleotide of the present invention, comprising a nucleic acid sequence selected from the group consisting of SEQ ID NO:24, SEQ ID NO:25, and SEQ ID NO:26.

In some embodiments, an antisense oligonucleotide comprising a nucleic acid sequence as set forth in SEQ ID NO:24 is used for reducing the production of membrane-anchored IgM immunoglobulins.

In some embodiments, an antisense oligonucleotide comprising a nucleic acid sequence as set forth in SEQ ID NO:25 is used for reducing the production of membrane-anchored IgG immunoglobulins.

In some embodiments, an antisense oligonucleotide comprising a nucleic acid sequence as set forth in SEQ ID NO:26 is used for reducing the production of membrane-anchored IgA immunoglobulins.

>IgM-PAM:
SEQ ID NO: 24
5′-TGTGCATATTTATTTCAGGGCAACA
>IgG-PAM:
SEQ ID NO: 25
5′-AGGATATTTTATTGTCATCACAAAA
>IgA-PAM:
SEQ ID NO: 26
5′-GGGCCACTTTATTGCACCTGGAAGG

The sequence of the ASO is selected so as to be specific, i.e. the ASO's are complementary only to the sequences of the pre-mRNA and not to other nucleic acid sequences.

In some embodiments, the antisense oligonucleotide of the present invention is stabilized. A “stabilized” ASO refers to an ASO that is relatively resistant to in vivo degradation (e.g. via an exo- or endo-nuclease). Stabilization can be a function of length or secondary structure. Alternatively, ASO stabilization can be accomplished via phosphate backbone modifications. Preferred stabilized ASO's of the instant invention have a modified backbone, e.g. have phosphorothioate linkages to provide maximal activity and protect the ASO from degradation by intracellular exo- and endo-nucleases. Other possible stabilizing modifications include phosphodiester modifications, combinations of phosphodiester and phosphorothioate modifications, methylphosphonate, methylphosphorothioate, phosphorodithioate, p-ethoxy, and combinations thereof. Chemically stabilized, modified versions of the ASO's also include “Morpholinos” (phosphorodiamidate morpholino oligomers, PMOs), 2′-O-Met oligomers, 2′Methoxy-ethyl oligomers, 2′-Fluoro (2′-F) oligomers, tricyclo (tc)-DNAs, U7 short nuclear (sn) RNAs, tricyclo-DNA-oligoantisense molecules (U.S. Provisional Patent Application Ser. No. 61/212,384 For: Tricyclo-DNA Antisense Oligonucleotides, Compositions and Methods for the Treatment of Disease, filed Apr. 10, 2009, the complete contents of which is hereby incorporated by reference, unlocked nucleic acid (UNA), locked nucleic acid (LNA), peptide nucleic acid (PNA), serinol nucleic acid (SNA), twisted intercalating nucleic acid (TINA), anhydrohexitol nucleic acid (HNA), cyclohexenyl nucleic acid (CeNA), D-altritol nucleic acid (ANA) and morpholino nucleic acid (MNA) have also been investigated in splice modulation. Recently, nucleobase-modified AOs containing 2-thioribothymidine, and 5-(phenyltriazol)-2-deoxyuridine nucleotides have been reported to induce exon skipping (Chen S, Le B T, Chakravarthy M, Kosbar T R, Veedu R N. Systematic evaluation of 2′-Fluoro modified chimeric antisense oligonucleotide-mediated exon skipping in vitro. Sci Rep. 2019 Apr. 15; 9(1):6078.). In some embodiments, the antisense oligonucleotides of the invention may be 2′-O-Me RNA/ENA chimera oligonucleotides (Takagi M, Yagi M, Ishibashi K, Takeshima Y, Surono A, Matsuo M, Koizumi M. Design of 2′-O-Me RNA/ENA chimera oligonucleotides to induce exon skipping in dystrophin pre-mRNA. Nucleic Acids Symp Ser (Oxf). 2004; (48):297-8). Other forms of ASOs that may be used to this effect are ASO sequences coupled to small nuclear RNA molecules such as U1 or U7 in combination with a viral transfer method based on, but not limited to, lentivirus or adeno-associated virus (Denti, M A, et al, 2008; Goyenvalle, A, et al, 2004). In some embodiments, the antisense oligonucleotides of the invention are 2′-O-methyl-phosphorothioate nucleotides.

The ASOs of the invention can be synthesized de novo using any of a number of procedures well known in the art. For example, the b-cyanoethyl phosphoramidite method (Beaucage et al., 1981); nucleoside H-phosphonate method (Garegg et al., 1986; Froehler et al., 1986, Garegg et al., 1986, Gaffney et al., 1988). These chemistries can be performed by a variety of automated nucleic acid synthesizers available in the market. These nucleic acids may be referred to as synthetic nucleic acids. Alternatively, ASO's can be produced on a large scale in plasmids (see Sambrook, et al., 1989). ASO's can be prepared from existing nucleic acid sequences using known techniques, such as those employing restriction enzymes, exonucleases or endonucleases. ASO's prepared in this manner may be referred to as isolated nucleic acids.

In some embodiments, the antisense oligonucleotide of the invention may be delivered in vivo alone or in association with a vector. In its broadest sense, a “vector” is any vehicle capable of facilitating the transfer of the antisense oligonucleotide of the invention to the cells. Preferably, the vector transports the nucleic acid to cells with reduced degradation relative to the extent of degradation that would result in the absence of the vector. In general, the vectors useful in the invention include, but are not limited to, naked plasmids, non-viral delivery systems (electroporation, sonoporation, cationic transfection agents, liposomes, nanoparticules, peptide-bound ASO, etc. . . . ), phagemids, viruses, other vehicles derived from viral or bacterial sources that have been manipulated by the insertion or incorporation of the antisense oligonucleotide nucleic acid sequences. Viral vectors are a preferred type of vector and include, but are not limited to nucleic acid sequences from the following viruses: RNA viruses such as a retrovirus (as for example moloney murine leukemia virus and lentiviral derived vectors), harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus. One can readily employ other vectors not named but known to the art. Typically, viral vectors according to the invention include adenoviruses and adeno-associated (AAV) viruses, which are DNA viruses that have already been approved for human use in gene therapy. Actually 12 different AAV serotypes (AAV1 to 12) are known, each with different tissue tropisms (Wu, Z Mol Ther 2006; 14:316-27). Recombinant AAV are derived from the dependent parvovirus AAV (Choi, V W J Virol 2005; 79:6801-07). The adeno-associated virus type 1 to 12 can be engineered to be replication deficient and is capable of infecting a wide range of cell types and species (Wu, Z Mol Ther 2006; 14:316-27). It further has advantages such as, heat and lipid solvent stability; high transduction frequencies in cells of diverse lineages, including hematopoietic cells; and lack of superinfection inhibition thus allowing multiple series of transductions. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion. Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by, intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation. In some embodiments, the antisense oligonucleotide nucleic acid sequence is under the control of a heterologous regulatory region, e.g., a heterologous promoter. The promoter can also be, e.g., a viral promoter, such as CMV promoter or any synthetic promoters.

As used herein, the term “subject”, refers to any mammals, such as a rodent, a feline, a canine, and a primate.

By reducing the amount of secreted immunoglobulins, the method of the present invention is particularly suitable for the treatment of diseases associated to autoimmunity or inflammation. Examples of said diseases include, but are not limited to arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, diabetic retinopathy, diabetic nephropathy, diabetic large-artery disorder, immune responses associated with acute and delayed hypersensitivity mediated by cytokines and T-lymphocytes, tuberculosis, sarcoidosis, granulomatosis including lymphomatoid granulomatosis, Wegener's granulomatosis, agranulocytosis, vasculitides, including vasculitis, large-vessel vasculitis (including polymyalgia rheumatica and giant-cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia pemiciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, thrombocytopenia (as developed by myocardial infarction patients, for example), including thrombotic thrombocytopenic purpura (TTP), post-transfusion purpura (PTP), heparin-induced thrombocytopenia, and autoimmune or immune-mediated thrombocytopenia such as idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant-cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine ophthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia areata, alopecia totalis, CREST syndrome (calcinosis, Raynaud's phenomenon, esophageal dysmotility, sclerodactyly, and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant-cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritis, reperfusion injury, ischemic reperfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, and endometriosis.

In some embodiments, the method for reducing the production of the secreted IgE immunoglobulins is particularly suitable for the treatment of IgE-mediated diseases. “The term “IgE-mediated diseases” includes atopic disorders, which are characterized by a general inherited propensity to respond immunologically to many common naturally occurring inhaled and ingested antigens and the continual production of IgE antibodies. Specific atopic disorders include allergic asthma, allergic rhinitis (conjunctivitis), atopic dermatitis, food allergy, anaphylaxis, contact dermatitis, allergic gastroenteropathy, allergic bronchopulmonary aspergillosis and allergic purpura (Henoch-Schönlein). Atopic patients often have multiple allergies, meaning that they have IgE antibodies to, and symptoms from, many environmental allergens, including seasonal, perennial and occupational allergens. Example seasonal allergens include pollens (e.g., grass, tree, rye, timothy, ragweed), while example perennial allergens include fungi (e.g., molds, mold spores), feathers, animal (e.g., pet or other animal dander) and insect (e.g., dust mite) debris. Example occupational allergens also include animal (e.g. mice) and plant antigens as well as drugs, detergents, metals and immunoenhancers such as isocyanates. Non-antigen specific stimuli that can result in an IgE-mediated reaction include infection, irritants such as smoke, combustion fumes, diesel exhaust particles and sulphur dioxide, exercise, cold and emotional stress. Specific hypersensitivity reactions in atopic and nonatopic individuals with a certain genetic background may result from exposure to proteins in foods (e.g., legumes, peanuts), venom (e.g., insect, snake), vaccines, hormones, antiserum, enzymes, latex, antibiotics, muscle relaxants, vitamins, cytotoxins, opiates, and polysaccharides such as dextrin, iron dextran and polygeline. Other disorders associated with elevated IgE levels, that appear to be IgE-mediated and are treatable with the method of the present invention include: ataxia-telangiectasia, Churg-Strauss Syndrome, eczema, enteritis, gastroenteropathy, graft-versus-host reaction, hyper-IgE (Job's) syndrome, hypersensitivity (e.g., anaphylactic hypersensitivity, candidiasis, vasculitis), IgE myeloma, inflammatory bowel disease (e.g., Crohn's disease, ulcerative colitis, indeterminate colitis and infectious colitis), mucositis (e.g., oral mucositis, gastrointestinal mucositis, nasal mucositis and proctitis), necrotizing enterocolitis and esophagitis, parasitic diseases (e.g., trypanosomiasis), hypersensitivity vasculitis, urticaria and Wiskott-Aldrich syndrome. Additionally, disorders that may be treatable by lowering IgE levels, regardless of whether the disorders themselves are associated with elevated IgE, and thus should be considered within the scope of “IgE-mediated disorder” include: Addison's disease (chronic adrenocortical insufficiency), alopecia, hereditary angioedema, anigioedema (Bannister's disease, angioneurotic edema), ankylosing spondylitis, aplastic anemia, arteritis, amyloidosis, immune disorders, such as autoimmune hemolytic anemia, autoimmune oophoritis, autoimmune orchitis, autoimmune polyendocrine failure, autoimmune hemolytic anemia, autoimmunocytopenia, autoimmune glomerulonephritis, Behcet's disease, bronchitis, Buerger's disease, bullous pemphigoid, Caplan's syndrome (rheumatoid pneumoconiosis), carditis, celiac sprue, Chediak-Higashi syndrome, chronic obstructive lung Disease (COPD), Cogan-Reese syndrome (iridocorneal endothelial syndrome), CREST syndrome, dermatitis herpetiformis (Duhring's disease), diabetes mellitus, eosinophilic fasciitis, eosinophilic nephritis, episcleritis, extrinsic allergic alveolitis, familial paroxysmal polyserositis, Felty's syndrome, fibrosing alveolitis, glomerulonephritis, Goodpasture's syndrome, granulocytopenia, granuloma, granulomatosis, granuloma myositis, Graves' disease, Guillain-Barre syndrome (polyneuritis), Hashimoto's thyroiditis (lymphadenoid goiter), hemochromatosis, histocytosis, hypereosinophilic syndrome, irritable bowel syndrome, juvenile arthritis, keratitis, leprosy, lupus erythematosus, Lyell's disease, Lyme disease, mixed connective tissue disease, mononeuritis, mononeuritis multiplex, Muckle-Wells syndrome, mucocutaneous lymphoid syndrome (Kawasaki's disease), multicentric reticulohistiocystosis, multiple sclerosis, myasthenia gravis, mycosis fungoides, panninculitis, pemphigoid, pemphigus, pericarditis, polyneuritis, polyarteritis nodoas, psoriasis, psoriatic arthritis, pulmonary arthritis, pulmonary adenomatosis, pulmonary fibrosis, relapsing polychondritis, rheumatic fever, rheumatoid arthritis, rhinosinusitis (sinusitis), sarcoidosis, scleritis, sclerosing cholangitis, serum sickness, Sézary syndrome, Sjögren's syndrome, Stevens-Johnson syndrome, systemic mastocytosis, transplant rejection, thrombocytopenic purpura, thymic alymphoplasia, uveitis, vitiligo, Wegener's granulomatosis.

In some embodiments, by reducing the production of the membrane-anchored immunoglobulin (i.e. BCR), the method of the present invention is suitable for inducing the apoptosis of malignant B cells. Thus the method of the present invention is thus particularly suitable for the treatment of B cell malignancies. As used herein, the term “B-cell malignancy” includes any type of leukemia or lymphoma of B cells. The term “B cell lymphoma” refers to a cancer that arises in cells of the lymphatic system from B cells. B cells are white blood cells that develop from bone marrow and produce antibodies. They are also known as B lymphocytes. B-cell malignancies include, but are not limited to, non-Hodgkin's lymphoma, Burkitt's lymphoma, small lymphocytic lymphoma, primary effusion lymphoma, diffuse large B-cell lymphoma, splenic marginal zone lymphoma, MALT (mucosa-associated lymphoid tissue) lymphoma, hairy cell leukemia, chronic lymphocytic leukemia, B-cell prolymphocytic leukemia, B cell lymphomas (e.g. various forms of Hodgkin's disease, B cell non-Hodgkin's lymphoma (NHL) and related lymphomas (e.g. Waldenstrom's macroglobulinaemia (also called lymphoplasmacytic lymphoma or immunocytoma) or central nervous system lymphomas), leukemias (e.g. acute lymphoblastic leukemia (ALL), chronic lymphocytic leukemia (CLL; also termed B cell chronic lymphocytic leukemia BCLL), hairy cell leukemia and chronic myoblastic leukemia) and myelomas (e.g. multiple myeloma). Additional B cell malignancies include, lymphoplasmacytic lymphoma, plasma cell myeloma, solitary plasmacytoma of bone, extraosseous plasmacytoma, extra-nodal marginal zone B cell lymphoma of mucosa-associated (MALT) lymphoid tissue, nodal marginal zone B cell lymphoma, follicular lymphoma, mantle cell lymphoma, mediastinal (thymic) large B cell lymphoma, intravascular large B cell lymphoma, primary effusion lymphoma, Burkitt's lymphoma/leukemia, grey zone lymphoma, B cell proliferations of uncertain malignant potential, lymphomatoid granulomatosis, and post-transplant lymphoproliferative disorder.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

A “therapeutically effective amount” is intended for a minimal amount of the active agent (i.e the ASO of the present invention) which is necessary to impart therapeutic benefit to a subject. For example, a “therapeutically effective amount” to a subject is such an amount which induces, ameliorates or otherwise causes an improvement in the pathological symptoms, disease progression or physiological conditions associated with or resistance to succumbing to a disorder. It will be understood that the total daily usage of the compounds of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically, the antisense oligonucleotide of the present invention is administered in the form of a pharmaceutical composition. Pharmaceutical compositions of the present invention may also include a pharmaceutically or physiologically acceptable carrier such as saline, sodium phosphate, etc. The compositions will generally be in the form of a liquid, although this need not always be the case. Suitable carriers, excipients and diluents include lactose, dextrose, sucrose, sorbitol, mannitol, starches, gum acacia, calcium phosphates, alginate, tragacanth, gelatin, calcium silicate, microcrystalline cellulose, polyvinylpyrrolidone, celluose, water syrup, methyl cellulose, methyl and propylhydroxybenzoates, mineral oil, etc. The formulations can also include lubricating agents, wetting agents, emulsifying agents, preservatives, buffering agents, etc. Those of skill in the art will also recognize that nucleic acids are often delivered in conjunction with lipids (e.g. cationic lipids or neutral lipids, or mixtures of these), frequently in the form of liposomes or other suitable micro- or nano-structured material (e.g. micelles, lipocomplexes, dendrimers, emulsions, cubic phases, nanoparticules, etc.).

The invention will be further illustrated by the following figures and examples. However, these examples and figures should not be interpreted in any way as limiting the scope of the present invention.

### FIGURES

FIG. 1. Decreased IgE production upon passive administration of ASO targeting the secreted immunoglobulin poly-adenylation signal (PAS) sequence. A. U266 cells were treated with 604 Vivo-Morpholino Standard-Control ASO (CTRL) or IgE-PAS targeting ASO for 48 hours. B. Specific secreted IgE RT-qPCR normalized on untreated cells was performed on 48 hours total RNA, n=7. C. Total IgE ImmunoCAP assay showing IgE production in culture supernatants, n=7. D. Reads exon coverage aligned on IGHE gene. Representative alignment of n=4 per group. Student T-test, **p<0.01, ****p<0.0001.

FIG. 2. Drastic decrease of IgE production upon treatment of primary cells secreting human IgE. InEps mice has been described previously (Laffleur B et al, Cell Reports 2015). Spleen cells were cultivated at 1×10⁶ cells/mL for 3 days with 1 μg/mL LPS and treated at day 1 with 1 μM hydroxy-tamoxifen and 204 Vivo-Morpholino Standard-Control ASO (CTRL) or IgE-PAS targeting ASO for 48 hours. A. Representation of InEps mice Igh locus, these mice were breeded with CreERT2 mice. Cre recombination is allowed by hydroxy-tamoxifen in-vitro treatment. B. Specific secreted-IgE and membrane-IgE RT-qPCR normalized on cells treated with hydroxy-tamoxifen only were performed on day 3 total RNA, n=5. C. B220+ cells were analyzed by flow cytometry at day 3 for intracellular-IgE expression D. B220+ IgE+ Cells analyzed by flow cytometry are significantly decreased upon ASO treatment, n=3 E. Total IgE ImmunoCAP assay showing IgE production in culture supernatants n=5. Student T-test, *p<0.05, **p<0.01, ****p<0.0001.

FIG. 3. Decrease allergen-specific IgE expression upon treatment of InEps hybridoma cells. Hybridomas (allergen-specific InEps B cells merged with SP2/0 cell line) were kindly provided by Dr A. Cuvillier (B-Cell Design company) and cultured for 48 hours with 304 Vivo-Morpholino Standard-Control ASO (CTRL) or IgE-PAS targeting ASO. A. Total IgE ImmunoCAP assay showing IgE production in culture supernatants, n=4. B. Specific secreted IgE RT-qPCR normalized on control treated cells was performed on 48 hours total RNA, n=4. C. Specific membrane-anchored IgE RT-qPCR normalized on control treated cells was performed on 48 hours total RNA, n=4. Student T-test, ns. non-significant,*p<0.05, ***p<0.001, ****p<0.0001.

FIG. 4. Design of specific human Ig-PAS ASOs. Specific human Ig-PAS ASO sequences and Ig PAS sequence alignments. Alignments show a 100% homology between IgA1 and IgA2. IgG-PAS ASO is covering IgG1, IgG2, IgG3 and IgG4 sequence with 100% homology, avoiding mismatch between these 4 sequences. IgE, IgM, IgA and IgG-PAS sequences don't show a significant similarity between one another. Ig PAS sequences were collected from NCBI database, GRCh38.p12 assembly, 20 base pairs upstream to 20 base pairs downstream from secreted Ig PAS on IGH locus. Multiple sequence alignments were made using MView bioinformatic tool.

FIG. 5. Design of specific human Ig-PAM ASOs. Specific human Ig-PAM ASO sequences and Ig PAM sequence alignments. Ig PAM sequences were collected from NCBI database, GRCh38.p12 assembly, 20 base pairs upstream to 20 base pairs downstream from secreted Ig PAM on IGH locus. Multiple sequence alignments were made using MView bioinformatic tool.

FIG. 6. Decreased membrane IgA expression upon passive administration of ASO targeting the membrane immunoglobulin poly-adenylation signal (PAM) sequence. A. AMO-1 cells were treated with 304 Vivo-Morpholino Standard-Control ASO (CTRL) or IgA-PAM targeting ASO for 48 hours and membrane IgA expression was measured by RT-qPCR on total RNA. B. IgA #6 hybridoma cells derived from transgenic mice expressing humanized IgA1 were treated with 604 Vivo-Morpholino Standard-Control ASO (CTRL) or IgA-PAM targeting ASO for 48 hours and membrane IgA expression was measured by RT-qPCR on total RNA

### EXAMPLE

Methods

ASO Design

Secreted immunoglobulin polyadenylation signal sequences (PAS) were collected from NCBI, GRCh38.p12 assembly, 20 base pairs upstream to 20 base pairs downstream from secreted Ig PAS on IGH locus. Multiple sequence alignments were made using MView bioinformatic tool. Vivo-morpholino IgE-PAS ASO (5′-GCTCTGAGCAGGCACAGTTTATTG-3′) (SEQ ID NO:14), Vivo-morpholino IgA-PAM ASO (5′-GGGCCACTTTATTGCACCTGGAAGG-3′) (SEQ ID NO:26) and irrelevant VivoStandard Control ASO (5′-CCTCTTACCTCAGTTACAATTTATA-3′) (SEQ ID NO:27) were designed and purchased at Gene Tools, LLC. ASO stock solutions were made at 5 mM with sterile nuclease-free water.

Mice

The InEps mouse model, harboring an insertion of a floxed human Cμ gene followed by a human Cε gene at the IgH locus, has been previously described (Laffleur et al, Cell Reports 2015). These mice were crossed with CreERT2 mice in order to induce Tamoxifen dependent expression of human IgE. Nine- to ten-month-old mice were used in all experiments and maintained in our animal facilities, at 21-23° C. with a 12-h light/dark cycle. Experiments were performed according to the guidelines of our institutional review board for animal experimentation (No. CREEAL 6-07-2012).

Cell Culture and ASO Treatments

U266 cell line was cultivated in RPMI1640 medium with Ultraglutamine and 20% FBS at 37° C. with 5% CO₂ and treated for 48 hours with 6 μM ASO. AMO-1 cell line was cultivated in RPMI1640 medium with Ultraglutamine and 10% FBS at 37° C. with 5% CO2 and treated for 48 hours with 3 μM ASO. Allergen-specific InEps hybridoma cell lines (anti-Penicillin, anti-Wasp Venom and anti-Ovalbumin) and IgA #6 hybridoma cells lines were kindly provided by Dr A. Cuvillier (B-Cell Design company) and cultured for 48 hours in DMEM Glutamax, high glucose medium 10% FBS at 37° C. with 5% CO₂ with 3 μM ASO (InEps) or 6 μM (IgA #6) ASO. Splenic B cells isolated from InEps/CreERT2+ mice were stimulated (1×10⁶ cells/ml) with 1 μg/ml lipopolysaccharide (LPS) (LPS-EB Ultrapure; InvivoGen) in RPMI 1640 medium with 10% FBS. At day 1, cells were treated for 48 hours with 1 μM Hydroxy-tamoxifen (Sigma H7904) and 2 μM ASO.

Flow Cytometry

InEps spleen cells were briefly washed with Trypsin-EDTA 0.05% (Gibco) to remove passively bound IgE before staining. Intracellular IgE staining was performed with IntraPrep Permeabilizaton Reagent (Beckman Coulter), using FITC anti-human IgE (A80-108F Bethyl Laboratories) and BV421 anti-mouse B220 (RA3-6B2 BioLegend). Data were acquired on a Beckton Dickinson LSRII Fortessa cytometer and analyzed with FlowLogic software.

Immuno-Assays

IgE concentrations were determined in culture supernatants by Total IgE ImmunoCap (ThermoFisher Diagnostic) assay on a Phadia250 instrument.

RT-qPCRs

Total RNA was prepared using Tri-reagent (Invitrogen) procedures. RT-PCR was carried out on 1 μg DNase I (Invitrogen)-treated RNA using High-Capacity cDNA Reverse Transcription Kit (Applied Biosystem). Priming for reverse transcription was done with Oligo(dT)₂₀ (Invitrogen). Quantitative PCRs were performed on cDNA samples equivalent to 5-10 ng of RNA per reaction, using SYBR® Premix Ex Taq™ (Tli RNaseH Plus), ROX plus or Premix Ex Taq™ (Probe qPCR), ROX plus (Takara) on a StepOnePlus Real-Time PCR system (Applied Biosystems). Transcripts were quantified according to the standard 2^{-ΔΔct} method after normalization to GAPDH (Hs02758991_g1 ThermoFisher Scientific Probe) or Gapdh (Mm99999915_g1 ThermoFisher Scientific Probe). For the quantification of secreted-IgE mRNA amounts, SYBR quantitative PCR were performed with forward 5′-CGAGCGGTGTCTGTAAATCC (SEQ ID NO:28) and reverse 5′-CACTGCACAGCTGGATGG (SEQ ID NO:29) primers. Membrane-IgE transcript mRNAs were quantified using Taqman quantitative PCR with an M1-M2 exon spanning probe designed and purchased at ThermoFisher Scientific. For the quantification of membrane-IgA mRNA amounts, SYBR quantitative PCR were performed with forward 5′-CCTTCGCTGTGACCAGCATA (SEQ ID NO:30) and reverse 5′-GTCCAGCACCACATAGGGAG (SEQ ID NO:31) primers.

Exon Coverage Analysis

Poly-adenylated mRNA-sequencing was performed on the Illumina NextSeq500 at the Functional Genomics Platform of Nice (Valbonne-Sophia-Antipolis, France). Reads were aligned with STAR on the hg38 genome version during the primary analysis. IGHE reads visualization was performed on IGV software.

Statistical Analysis

The results are expressed as the mean±standard error of the mean (SEM), and overall differences between variables were evaluated by Student t test using Prism GraphPad software (San Diego, Calif.).

Results

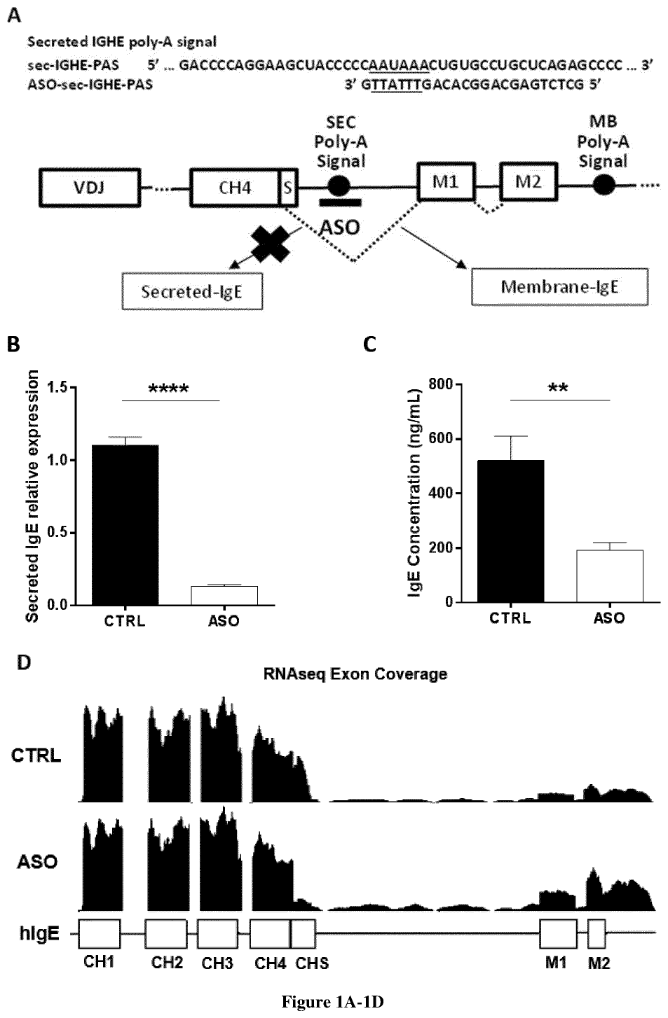

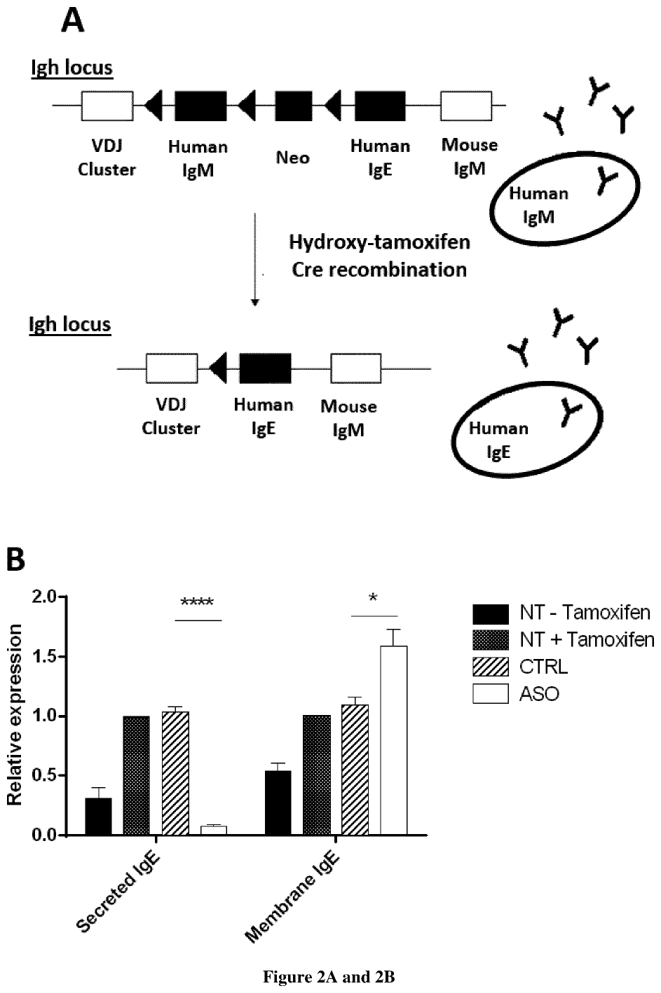

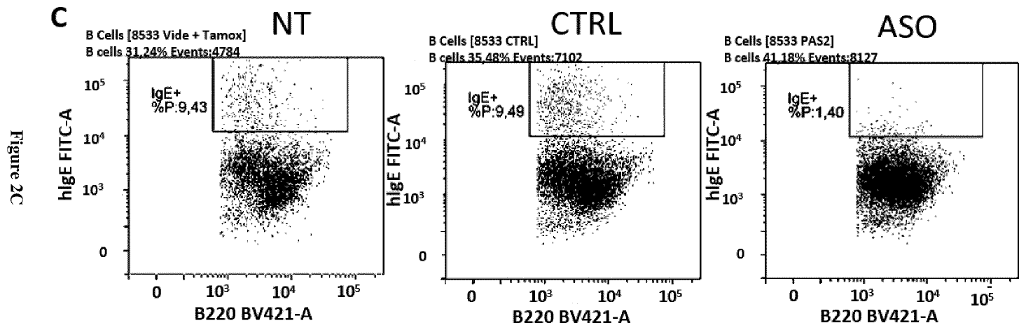

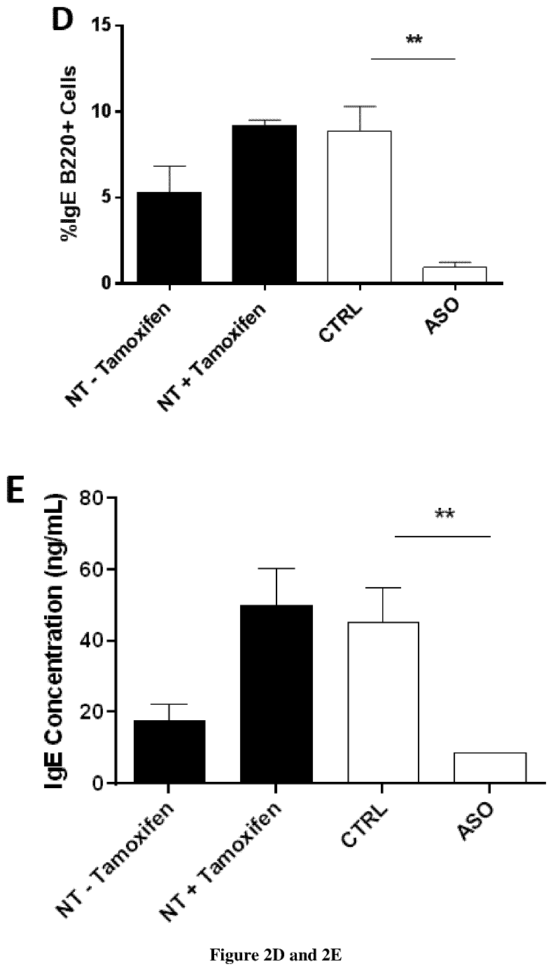

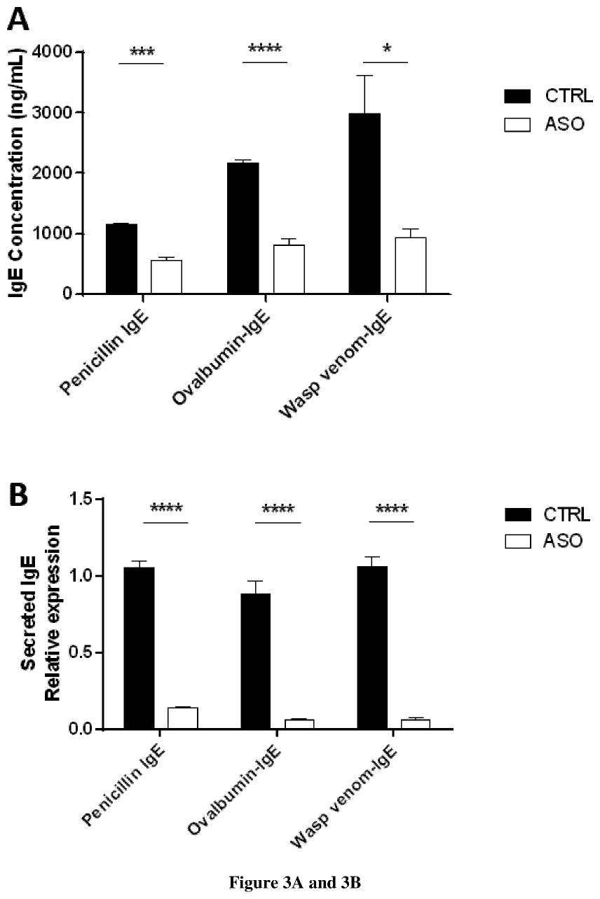

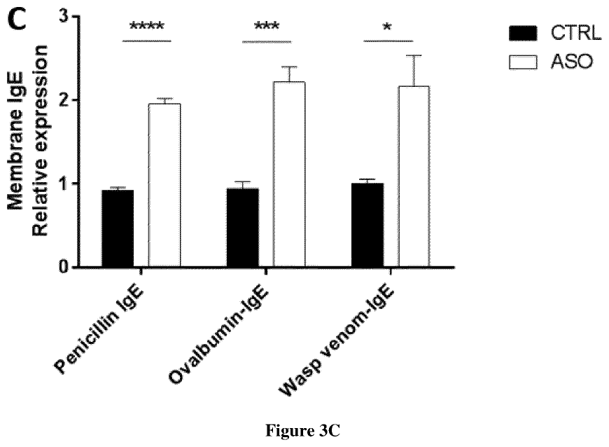

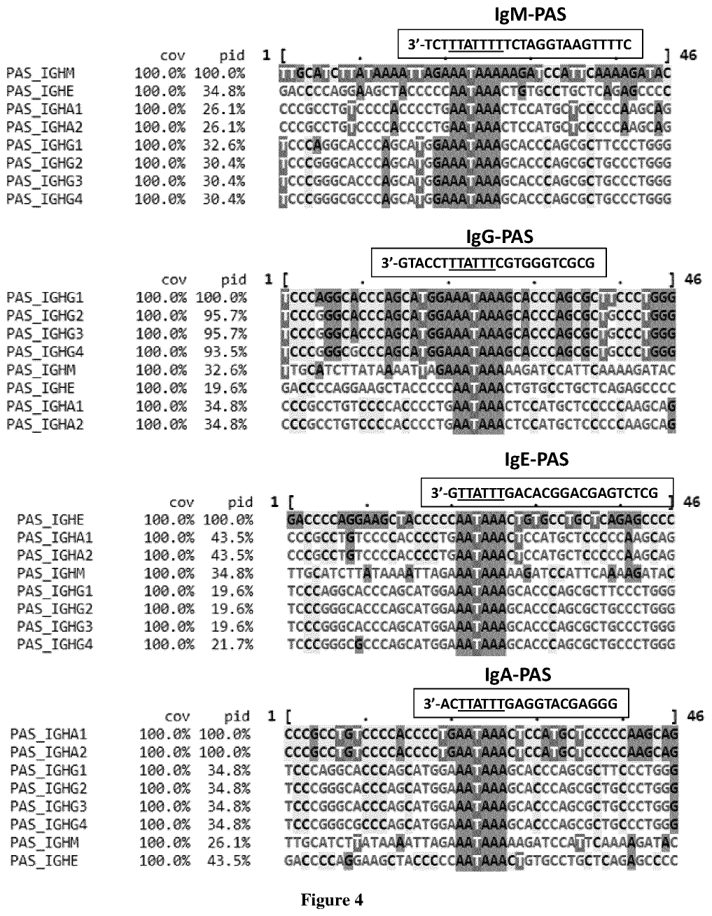

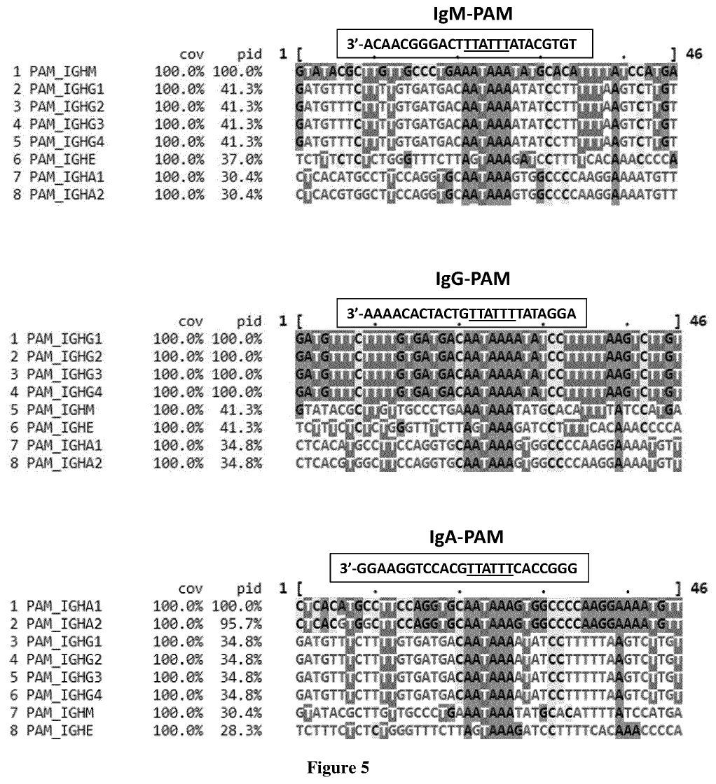

Immunoglobulins (Ig) are expressed either on the surface of B cells or as secreted antibodies by plasma cells that represents the final stage of B cell differentiation. The proof of concept has been obtained using an ASO hybridizing to the polyadenylation signal (PAS) sequence of the transcript encoding the secreted form of IgE. Indeed, the targeting of this PAS sequence induces a drastic decrease in IgE production (FIGS. 1A-1D, FIGS. 2A-2E and FIGS. 3A-3C). The particular configuration of the Ig heavy (IGH) chain locus makes it possible to mask the secreted poly-A site (PAS) of a particular subclass of Ig while promoting the use of an alternative polyadenylation signal encoding the membrane form of the corresponding Ig (FIG. 4). In the case of IgE, a previous study by the team demonstrated that membrane-anchored IgE expression has a pro-apoptotic effect in B cells (Laffleur et al, Cell Reports 2015). The overproduction of allergen-specific secreted IgE is one of the established features of many forms of allergies including chronic allergic asthma or some food or skin allergies (Platts-Mills et al J Allergy Clin Immunol 2016). This invention could be extended to other Ig subclasses (i.e. IgM, IgG or IgA) and to membrane-anchored polyadenylation signals (PAM), and hence, should have broad clinical applications in B-cell malignancies and antibody-mediated pathologies (FIG. 5).

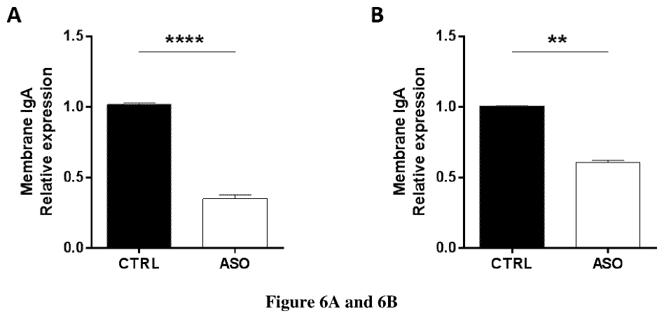

FIGS. 6A and 6B show decreased membrane IgA expression upon passive administration of ASO targeting the membrane immunoglobulin poly-adenylation signal (PAM) sequence.

### REFERENCES

Throughout this application, various references describe the state of the art to which this invention pertains. The disclosures of these references are hereby incorporated by reference into the present disclosure.

1. A method of modulating the expression of an immunoglobulin in a subject in need thereof comprising administering to the subject an effective amount of:
an antisense oligonucleotide complementary to a sequence comprising the secretory polyadenylation signal within the pre-mRNA molecule encoding for the immunoglobulin heavy chain for reducing the production of the secreted immunoglobulin or
an antisense oligonucleotide complementary to a sequence comprising the membrane-anchored specific polyadenylation signal within the pre-mRNA molecule encoding for the immunoglobulin heavy chain for reducing the production of the membrane-anchored immunoglobulin.

2. The method of claim 1 wherein the immunoglobulin is an IgG, IgA, IgM, or IgE.

3. The method of claim 1 wherein the antisense oligonucleotide comprises the sequence as set forth in SEQ ID NO:2 or the sequence as set forth in SEQ ID NO:3.

4. The method of claim 1 wherein the antisense oligonucleotide has a length of at least 15 nucleotides.

5. The method of claim 4 wherein the antisense oligonucleotide has a length of 15, 16, 17, 18, 19, 20, 21, 22, 23, 24, 25, 26, 27, 28, 29, or 30 nucleotides.

6. The method of claim 1 wherein the antisense oligonucleotide is complementary to the sequence as set forth in SEQ ID NO:4, SEQ ID NO:5, SEQ ID NO:6, SEQ ID NO:7, SEQ ID NO:8, SEQ ID NO:9, SEQ ID NO:10, or SEQ ID NO:11.

7. The method of claim 6 wherein the antisense oligonucleotide comprises a nucleic acid sequence selected from the group consisting of SEQ ID NO:12, SEQ ID NO:13, SEQ ID NO:14 and SEQ ID NO:15.

8. The method of claim 1 wherein the antisense oligonucleotide is complementary to the sequence as set forth in SEQ ID NO:16, SEQ ID NO:17, SEQ ID NO:18, SEQ ID NO:19, SEQ ID NO:20, SEQ ID NO:21, SEQ ID NO:22, or SEQ ID NO:23.

9. The method of claim 8 wherein the antisense oligonucleotide comprises a nucleic acid sequence selected from the group consisting of SEQ ID NO:24, SEQ ID NO:25, and SEQ ID NO:26.

10. The method of claim 1 wherein the antisense oligonucleotide is stabilized.

11. The method of claim 1 wherein the subject suffers from a disease associated to B-cell development.

12. The method of claim 11 wherein the subject suffers form autoimmunity or inflammation.

13. The method of claim 1 wherein the subject suffers from an IgE-mediated disease.

14. The method of claim 1 wherein the subject suffers from a B cell malignancy.

15. An antisense oligonucleotide comprising a nucleic acid sequence selected from the group consisting of SEQ ID NO:12, SEQ ID NO:13, SEQ ID NO:14, SEQ ID NO:15, SEQ ID NO:24, SEQ ID NO:25, and SEQ ID NO:26.

16. A pharmaceutical composition comprising the antisense oligonucleotide of claim 15.


SEQUENCE LISTING (31 sequences)

SEQ ID NO: 1 - RNA, 6 nt, Homo sapiens
  misc_feature (2)..(2): N is A or G
anuaaa

SEQ ID NO: 2 - DNA, 6 bp, Artificial
  consensus motif
  misc_feature (5)..(5): N is T or C
tttant

SEQ ID NO: 3 - RNA, 6 nt, Artificial
  consensus motif
  misc_feature (5)..(5): N is U or C
uuuanu

SEQ ID NO: 4 - DNA, 46 bp, Homo sapiens
cccgcctgtccccacccctgaataaactccatgctcccccaagcag

SEQ ID NO: 5 - DNA, 46 bp, Homo sapiens; identical to SEQ ID NO: 4

SEQ ID NO: 6 - DNA, 46 bp, Homo sapiens
tcccaggcacccagcatggaaataaagcacccagcgcttccctggg

SEQ ID NO: 7 - DNA, 46 bp, Homo sapiens
tcccgggcacccagcatggaaataaagcacccagcgctgccctggg

SEQ ID NO: 8 - DNA, 46 bp, Homo sapiens; identical to SEQ ID NO: 7

SEQ ID NO: 9 - DNA, 46 bp, Homo sapiens
tcccgggcgcccagcatggaaataaagcacccagcgctgccctggg

SEQ ID NO: 10 - DNA, 46 bp, Homo sapiens
ttgcatcttataaaattagaaataaaaagatccattcaaaagatac

SEQ ID NO: 11 - DNA, 46 bp, Homo sapiens
gaccccaggaagctacccccaataaactgtgcctgctcagagcccc

SEQ ID NO: 12 - DNA, 25 bp, Artificial
  IgM-PAS ASO
cttttgaatggatctttttatttct

SEQ ID NO: 13 - DNA, 23 bp, Artificial
  IgG-PAS ASO
gcgctgggtgctttatttccatg

SEQ ID NO: 14 - DNA, 24 bp, Artificial
  IgE-PAS ASO
gctctgagcaggcacagtttattg

SEQ ID NO: 15 - DNA, 20 bp, Artificial
  IgA-PAS ASO
gggagcatggagtttattca

SEQ ID NO: 16 - DNA, 46 bp, Homo sapiens
ctcacatgccttccaggtgcaataaagtggccccaaggaaaatgtt

SEQ ID NO: 17 - DNA, 46 bp, Homo sapiens
ctcacgtggcttccaggtgcaataaagtggccccaaggaaaatgtt

SEQ ID NO: 18 - DNA, 46 bp, Homo sapiens
gatgtttcttttgtgatgacaataaaatatcctttttaagtcttgt

SEQ ID NO: 19 - DNA, 46 bp, Homo sapiens; identical to SEQ ID NO: 18

SEQ ID NO: 20 - DNA, 46 bp, Homo sapiens; identical to SEQ ID NO: 18

SEQ ID NO: 21 - DNA, 46 bp, Homo sapiens; identical to SEQ ID NO: 18

SEQ ID NO: 22 - DNA, 46 bp, Homo sapiens
gtatacgcttgttgccctgaaataaatatgcacattttatccatga

SEQ ID NO: 23 - DNA, 46 bp, Homo sapiens
tctttctctctgggtttcttagtaaagatccttttcacaaacccca

SEQ ID NO: 24 - DNA, 25 bp, Artificial
  IgM-PAM ASO
tgtgcatatttatttcagggcaaca

SEQ ID NO: 25 - DNA, 25 bp, Artificial
  IgG-PAM ASO
aggatattttattgtcatcacaaaa

SEQ ID NO: 26 - DNA, 25 bp, Artificial
  IgA-PAM ASO
gggccactttattgcacctggaagg

SEQ ID NO: 27 - DNA, 25 bp, Artificial
  control ASO
cctcttacctcagttacaatttata

SEQ ID NO: 28 - DNA, 20 bp, Artificial
  primer
cgagcggtgtctgtaaatcc

SEQ ID NO: 29 - DNA, 18 bp, Artificial
  primer
cactgcacagctggatgg

SEQ ID NO: 30 - DNA, 20 bp, Artificial Sequence
  primer
ccttcgctgtgaccagcata

SEQ ID NO: 31 - DNA, 20 bp, Artificial Sequence
  primer
gtccagcaccacatagggag